(19) United States
(12) Patent Application Publication
(10) Pub. No.: US 2007/0264656 A1
(43) Pub. Date: Nov. 15, 2007

(54) METHOD OF MANUFACTURING PROBE-IMMOBILIZED CARRIER

(75) Inventor: Masashi Kawamura, Tokyo (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 11/741,281

(22) Filed: Apr. 27, 2007

(30) Foreign Application Priority Data
May 15, 2006 (JP) 2006-135029

(51) Int. Cl. C12Q 1/68

(52) U.S. Cl. 435/6; 427/2.11

(57) ABSTRACT

A probe-immobilized carrier in which a probe capable of specifically binding to a target is immobilized on a substrate is manufactured by a method comprising the steps of: (1) coating the substrate with a reactive substance having a reactive group for immobilizing the probe on the substrate; (2) applying the probe to a surface of the substrate coated with the reactive substance; (3) immobilizing the applied probe on the substrate; and (4) applying an inactivating compound capable of inactivating the probe to a probe-immobilized area on the substrate to inactivate an unreacted probe remaining on the substrate in the probe-immobilized area.

### BACKGROUND OF THE INVENTION

1. Field of the Invention

The present invention relates to a method of manufacturing a probe-immobilized carrier by immobilizing a probe for detecting a target substance on a substrate.

2. Related Background Art

A method using a probe-immobilized carrier, in which a probe is immobilized on a substrate, has been known as one of technologies for quickly and precisely determining a base sequence of a nucleic acid, detecting a nucleic acid having a specific target base sequence in a specimen, and identifying various bacterial species. The probe to be immobilized on the substrate is a substance that specifically binds to a target substance by a hybridization reaction, a substrate-enzyme reaction, an antigen-antibody reaction, etc. Examples of the probe-immobilized substrate include a probe array and a DNA chip in which a plurality of different probes are spotted and immobilized on the substrate so that the plurality of spots are aligned on the substrate.

Various methods have been known as those for immobilizing probes on substrates. Example thereof includes a method involving immobilizing probes on a substrate by sequentially synthesizing the probes on the substrate (i.e., on-chip method). Another method involves placing previously-prepared probes on a substrate using pins, a stamp, or the like and then immobilizing the probes on the substrate.

U.S. Pat. No. 5,143,854 discloses a specific example of the sequential synthesis method. In this method, a protective group is removed from a selected area of a substrate by an activator. Subsequently, a monomer having a removable protective group is then applied on the selected area of the substrate being activated by the removal of the protective group. Further, the removal of a protective group and the monomer binding are repeated to synthesize polymers (polynucleotides) having various sequences on the substrate.

Japanese Patent Application Laid-Open No. 08-23975 discloses a method of immobilizing a biologically active substance on a substrate using a polymeric compound having a carbodiimide group. In other words, the method conducts the immobilization such that the polymeric compound having the carbodiimide group, which is a reactive group carried on the substrate, is brought into contact with the substance having an active group to be coupled with the carbodiimide group. Further, Japanese Patent Application Laid-Open No. 2001-178442 discloses a method of immobilizing a DNA fragment on the surface of a solid-phase carrier by using a thiol group. In this method, the DNA fragment, which has a thiol group at a terminal thereof, is dropped on a substrate having an immobilized linear molecule with a reactive group capable of reacting with and covalently binding to the thiol group. As a result, the DNA fragment is immobilized on the surface of the solid-phase carrier as the DNA fragment is covalently bound to the linear molecule. In contrast, Japanese Patent Application Laid-Open No. 2000-295990 discloses a technology for binding of a DNA fragment on a substrate, where an aqueous solution is prepared by dissolving or dispersing the DNA fragment and a hydrophilic polymer in an aqueous medium and then spotted on the substrate to bind the DNA fragment on the substrate.

As described above, a probe having an active group is prepared, while a reactive substance having a reactive group capable of binding to the active group is applied on a substrate. Subsequently, the probe is applied on the substrate, thereby resulting in binding of the probe to the substrate. A probe array, in which probes are disposed on a substrate by spot-immobilization, is generally desired to be of high sensitivity. This is because a decrease in detection accuracy occurs as the S/N ratio decreases when the absolute amount of a target substance to be detected by the probe array is small.

In general, a probe to be applied to a substrate is being dispersed or dissolved in a certain liquid and the resulting solution is then applied and immobilized on the substrate to form spots. Therefore, there is an idea of providing a method of improving the sensitivity of the probe array by increasing the concentration of the probe in the solution to increase the content of the probe per spot formed. In this case, however, the probe may be excessively supplied relative to a substance provided on the substrate to bind to the probe, when the spot is formed by the high-concentrated probe solution.

As shown in FIG. 4, an unreacted probe 401 not bound to the substrate may remain in a liquid droplet when the liquid containing the probe is provided on the substrate. Therefore, two problems occur as follows when the immobilized spot is washed by a liquid phase treatment with water, a detergent, or the like.

The first problem is that, when the probe-immobilized carrier is washed after forming a spot on a substrate 503 as shown in FIG. 5, the unreacted probe 401 may flow to an area other than the area on which the spot is formed (spot area 501), thereby allowing the unreacted probe 401 to be applied to a background area 502 on the substrate 503. When the unreacted probe 401 binds to a reactive substance coated on the area other than the spot area 501 of the substrate 503, the spot may become irregular in shape or the area between the adjacent two adjacent spots may collapse, and thus the boundary between the spot and the background 502 may be hardly distinguished, thereby leading to a decrease in detection accuracy. In addition, even in the case of a probe-immobilized carrier that does not require a washing step after forming a spot, an unreacted probe stacked on the spot area 501 may flow to the background area 502 when the liquid containing the target substance is brought into contact with the unreacted probe, thereby leading to a similar problem with the above.

The second problem is that, when spots of different kinds of probes are formed on a substrate, an unreacted probe may invade the spot area 501 of a different probe. For instance, when a liquid droplet is removed by washing in a liquid phase while the unreacted probe remains in the droplet spotted on the substrate, the probe flown out by washing may contaminate the adjacent spot area 501. As a result, the spot area 501 on which only one kind of the probe is to be immobilized is provided with another kind of the probe, so a normal detection cannot be performed.

Conventionally, attempts have been made to provide a method of preventing a target substance from being adsorbed on the background area 502 of the probe-immobilized carrier. For preventing the adsorption of the target substance to the background area 502, a blocking treatment using skim milk or the like is known in the art. Further, it is also known that a blocking treatment is carried out by dipping the substrate into a water-soluble polymeric solution after immobilizing the probe on the substrate. Specifically, as described in Japanese Patent No. 2794728, there is a method of immobilizing a probe on a nitrocellulose film and then dipping the film into a solution containing PVA and/or PVP to carry out a blocking treatment.

However, when the concentration of the probe in the spot is increased to the saturated concentration or more at which the probe is allowed to react with the reactive substance coated on the substrate to enhance the sensitivity, an unreacted probe can be found in the spot on the substrate even the above-mentioned blocking agent is used. The unreacted probe may flow out of the spot area 501 during the steps of blocking and washing. When the unreacted probe is immobilized on the background area 502 of the substrate, the effects of the blocking treatment on the background area 502 can be lowered.

### SUMMARY OF THE INVENTION

An object of the present invention is to solve the aforementioned problem inherent to the prior art as described above. In other words, the present invention intends to provide a method of manufacturing a probe-immobilized carrier for detecting a target substance, which can be prevented from contamination between spots and probe-immobilization on the background area 502 during the manufacture of the probe-immobilized carrier.

The present invention provides a method of manufacturing a probe-immobilized carrier in which a probe capable of specifically binding to a target is immobilized on a substrate substance, comprising the steps of: (1) coating the substrate with a reactive substance having a reactive group for immobilizing the probe on the substrate; (2) applying the probe to a surface of the substrate coated with the reactive substance; (3) immobilizing the applied probe on the substrate; and (4) applying an inactivating compound capable of inactivating the probe to a probe-immobilized area on the substrate to inactivate an unreacted probe remaining on the substrate in the probe-immobilized area.

The conventional blocking method using a blocking solution to be specifically adsorbed or bound on an area (background area) other than the spot area 501 on the probe substrate may cause the flow of an unreacted probe out of a spot formed on the substrate during the blocking step. The unreacted probe may be adsorbed on the background area 502 or in a spot containing different kinds of probes, thereby leading to a decrease in detection accuracy of the probe-immobilized carrier in many cases. However, according to the present invention, the unreacted probe remained in the spot formed is specifically inactivated. Therefore, the unreacted probe can be prevented from flowing out to an area other than the spot area 501 and being immobilized thereon.

Further features of the present invention will become apparent from the following description of exemplary embodiments with reference to the attached drawings.

### BRIEF DESCRIPTION OF THE DRAWINGS

FIG. 1 is a graph that illustrates an inactivation effect of an unreacted probe with Maleimido-PEGs.

FIG. 2 is a graph that illustrates an inactivation effect of an unreacted probe with Carboxyl-PEGs (NHS active ester).

FIG. 3 is a diagram that illustrates the outline of a method of manufacturing the probe-immobilized carrier of the present invention.

FIG. 4 is a diagram that illustrates the presence of an unreacted probe in a liquid droplet of a probe solution applied on a substrate.

FIG. 5 is a diagram that illustrates a spot area and a background area on the substrate.

### DESCRIPTION OF THE PREFERRED EMBODIMENTS

Preferred embodiments of the present invention will now be described in detail in accordance with the accompanying drawings

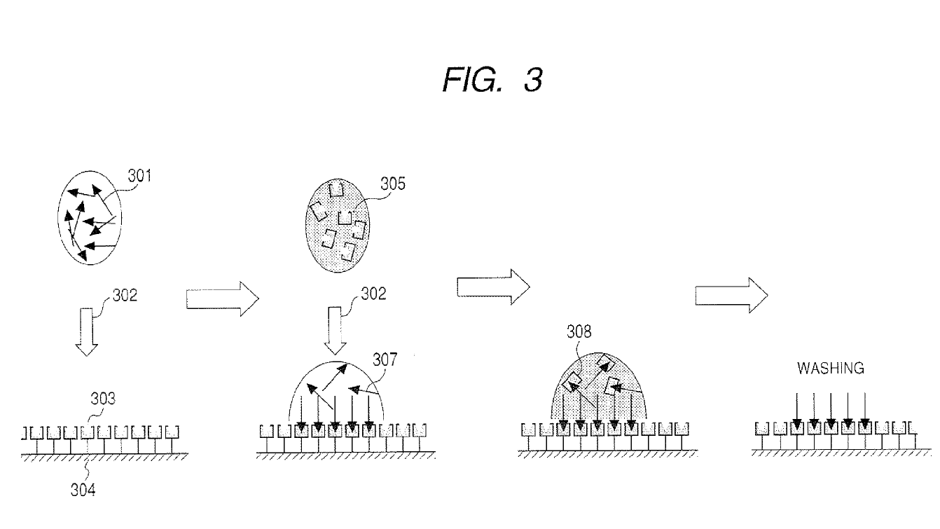

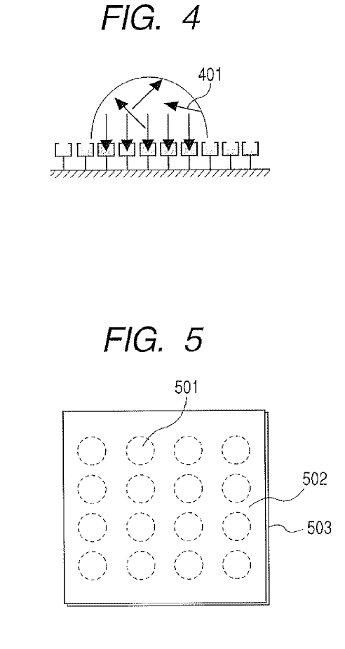

A method of manufacturing a probe-immobilized carrier of the present invention is schematically illustrated in FIG. 3. The characteristic feature of the present invention is that a liquid droplet 302 containing probes 301 applied on a substrate 304 is subjected to a treatment for inactivating an unreacted probe 307 in the liquid droplet 302. A spot is obtained by applying the droplet 302 containing the probes 307 to a certain area defined on the surface of the substrate 304 in such a manner that the resulting spot is formed into a desired shape of a strip, a spot, a dot, or the like.

Further, biological macromolecules such as proteins (including complex proteins), nucleic acids, sugar chains (including complex sugars), and lipids (including complex lipids) can be used as the probe 301. Specific examples thereof include enzymes, hormones, pheromones, antibodies, antigens, haptens, peptides, synthetic peptides, DNAs, synthetic DNAs, RNAs, synthetic RNAs, PNAs, synthetic PNAs, gangliosides, and lectins. The probe can be immobilized on the substrate by reacting the active group (X) on the probe side with the reactive group on the substrate side. The reactive group 303 of the substrate may be one inherently included in the substrate itself or one applied onto the substrate. Examples of the active group (X) on the probe side include a thiol group, an amino group, a maleimide group, an N-hydroxy-succinimidyl ester group, a formyl group, a carboxyl group, an acryl amide group, and an epoxy group.

Further, as far as the probe is one inherently having an active group (X), it may use its own active group (X). In contrast, the probe does not inherently have an active group (X) may be modified with any active group.

In addition, when a thiol group is introduced into a probe, for example when an automatically synthesized DNA is used as a probe, Thiol-Modifier (manufactured by Glen Research Corp.) can be used for the synthesis with an automated DNA synthesizer. Note that, the thiol group introduction method is not particularly limited thereto. On the other hand, when an amino group is introduced into a probe, for example, when an automatically synthesized DNA is used as a probe, Amino-Modifier (manufactured by Glen Research Corp.) can be used fox the synthesis with an automated DNA synthesizer. Note that, the amino group introduction method is not particularly limited thereto.

The substrate may be made of any material as far as it functions as a support for the probe to immobilize the probe thereon with no difficulty in detecting a target substance using a probe-immobilized carrier obtained. Exemplary materials include inorganic materials, such as glass, and polymer materials, such as various kinds of resins. In addition, for enhancing the sensitivity by increasing the density of the immobilized probe per unit area, the substrate used may be selected from porous materials having large surface areas, such as porous glass, paper, nitrocellulose, and acrylamide. Alternatively, the substrate may be any of various materials in which those porous materials are applied onto the surface of the material. Further, the material for the substrate may preferably be any of those in which an amino group, a maleimide group, an acrylamide group, an N-hydroxysuccinimidyl ester group, a formyl group, a carboxyl group, or an epoxy group is introduced on the surface of the material as a reactive group (Y) to react with the active group (X) on the probe side using a known method. Alternatively, the substrate may be selected from the materials inherently including these reactive groups. In addition, the reactive groups are not limited to those listed above and may be suitably selected depending on the active group (X) on the probe side. In the preferred combination of X and Y, for example, Y is an N-hydroxysuccinimidyl ester group when X is an amino group. In addition, Y is a maleimide group when X is a thiol group.

For forming a spot on a predetermined position on the substrate, a method of applying a liquid containing a probe to a predetermined position of a substrate, such as an aqueous solution of a probe, can be suitably used. The application of the liquid containing the probe onto the substrate can be performed using any of various methods which have been employed in the production of DNA array. Among them, a method that allows the liquid containing the probe to be spotted on the predetermined position on the substrate can be used. The spotting method may be a pipette method, a pin method, a pin & ring method, an inkjet method, or the like. Among them, the inkjet method is particularly suitable because it is able to precisely control the spotting of the liquid containing the probe to the predetermined position on the substrate and therefore to precisely arrange spots on the substrate with high density.

A spot-formed area on the surface of the substrate is referred to as a “spot area” 501 and an area other than the spot area 501 on the surface of the substrate is referred to as a “background area” 502. At the stage of forming a spot on the surface of the substrate, an unreacted probe, which does not bind to a reactive substance on the substrate, should be prevented from flowing out to the background area 502. Therefore, at least a treatment for inactivating the unreacted probe is performed on the spot formed on the substrate. Such inactivating treatment can preferably be carried out by applying the inactivating compound to the substrate.

For coating the substrate with the inactivating substance, though not shown in FIG. 3, a spotting method, a coating method, an atomizing method, a gas-phase contacting method, or the like can be used.

Among them, in particular, the pipette method, the pin method, the pin & ring method, the inkjet method, or the like can be used as the spotting method.

Among these spotting methods, the inkjet method is preferred because it is able to control the amount of a liquid to be applied on the substrate and a spotting position with high precision. An inactivating compound is applied on the spot area 501 depending on the positional information of the spot area 501 on the substrate. Therefore, the use of a minimum amount of the inactivating compound 305 can lead to a sufficient inactivating effect.

Hereinafter, as an example of the inactivating treatment, a method of applying a liquid 306 containing an inactivating substance to a substrate using the inkjet method will be discussed. In this case, the positional information of a spot formed is stored in a memory and the inactivating substance is then applied on the substrate by the inkjet method on the basis of the stored positional information, thereby efficiently coating the substrate with the inactivating compound 305.

A preferred embodiment of the inkjet method may be of applying the inactivating compound to the substrate by the steps (1) to (7) as follows.

(1) A probe solution or two or more different probe solutions and an inactivating-compound solution or two or more different inactivating-compound solutions are supplied to different reservoirs, respectively.

(2) The probe solution 302 is ejected on a predetermined probe-recording position on the substrate 304.

(3) The probe 301 is reacted with the substrate 304 for a sufficient time to complete the reaction at the optimal temperature.

(4) The solution 306 containing the inactivating compound is ejected on the substrate at the same position as that of the probe-recording position. Here, for surely carrying out the reaction of the probe with the inactivating compound, solvents used in both solutions may have solvent compositions, which can be mixed with each other, or may be the same solvent.

(5) An unreacted probe remaining on the substrate is reacted with the inactivating compound for a sufficient time to complete the reaction at the optimum temperature.

(6) The substrate is washed with a buffer or the like described below.

(7) If required, the substrate is dried by a spray method, a spin-dry method, or the like.

Here, a method of supplying the liquid to the reservoir of the above ejecting device is capable of correctly and efficiently supplying the liquid from a well plate in which the probe solution or the inactivating-compound solution is reserved to the reservoir.

Further, a transparent member may be used for part of an inkjet head and an alignment camera may be then placed above the position on which the inkjet head is installed in the vertical direction to effectively align the inkjet head and the substrate, thereby applying and stacking the inactivating compound on a spot precisely formed on the substrate. As the details thereof are described in Japanese Patent Application Laid-Open No. 2005-169805, the details thereof will be omitted herein.

Further, the use of a method described in Japanese Patent Application Laid-Open No. 2005-169795, the detailed description of which will be omitted, prevents variance in dot diameters, misdirection, and non-ejection, so that the method can be suitably used if needed. Obviously, when the present invention is carried out using the inkjet method, the alignment method and the procedures for preventing variance in dot diameters and non-ejection may be suitably employed, but not specifically limited to.

The solvent for dispersing or dissolving the probe or the solvent for dissolving the inactivating compound is selected from those that do not substantially affect on the desired functions of the probe or the inactivating compound. In particular, it is selected from those that do not substantially affect on the probe or the inactivating compound ejected from the inkjet head.

For example, when the inkjet head is a bubble-jet head having a mechanism for discharging a liquid from a nozzle with the application of thermal energy, a preferable component to be contained in the probe solvent is an aqueous liquid medium containing glycerin, thiodiglycol, isopropyl alcohol, and acetylene alcohol. Further, specifically, an aqueous liquid containing 5 to 10 wt % of glycerin, 5 to 10 wt % of thiodiglycol, and 0.5 to 1 wt % of acetylene alcohol is suitably used as a probe medium. In addition, when the inkjet head is a piezo-jet head that ejects a solution using a piezoelectric element, a preferable component to be contained in a probe solvent is a liquid containing ethylene glycol and isopropyl alcohol. More specifically, an aqueous solvent containing 5 to 10 wt % of ethylene glycol and 0.5 to 2 wt % of isopropyl alcohol is suitably used as a probe solvent.

By ejecting the probe solution using the probe solvent as described above from the inkjet head, a spot formed can be a round shape and the spot area 501 can be less spread out with flattened spot. In addition, even if the spots are formed in high density, the adjacent spots can be effectively prevented from joining together. Needless to say, the characteristics of the probe solution of the present invention are not limited to those described above.

Further, when the inactivating compound is applied on the spot formed, the liquid containing the inactivating compound may be applied so that the spot area 501 cannot be spread out more than necessary.

The reasons thereof will be as follows. When the formation of the spot and the application of the inactivating compound are carried out by spotting, the total amount of a liquid droplet containing the probe applied on the substrate and a liquid droplet containing the inactivating compound to be further applied from above the liquid droplet defines the final size of the spot. In this case, if the size of the spot is too large, the probe may be flown out to the background area 502 outside of the spot area 501 before the reaction of the unreacted probe with the inactivating compound occurs. Then, if the unreacted probe flown out is immobilized on the background area 502 (area other than the spot area 501 on the substrate), the size of the spot area 501 eventually becomes larger than the predetermined size. As a result, it may disturb the formation of spots on the substrate in high density.

For preventing the above-mentioned spot area 501 from spreading out more than necessary, a method of adjusting the amount of the liquid containing the inactive compound to be applied can be suitably employed. The formation of spots and the application of the inactivating compound are preferably defined so that the volumes of their liquid droplets can be equal to each other. More preferably, for substantially preventing the diameters of liquid droplets from being varied, the inactivating compound is set at high concentration while the amount of the liquid droplet containing the inactivating compound is reduced. Thus, the above-mentioned object can be attained. Here, when the amount of the reactive group in the inactivating compound is set to an equal mole or more with respect to the active group of the unreacted probe, the amount of the liquid droplet containing the inactivating compound can be reduced as far as a device for applying a minute liquid droplet can eject the liquid droplet. In this case, the concentration of the inactivating compound may be defined within the range of solubility to the solvent to be used.

Further, for entirely inactivating the active group of the unreacted probe as far as possible, the concentration of the inactivating substance in the liquid containing the inactivating compound may be sufficiently increased.

On the other hand, a method used for applying the inactivating compound to the substrate on which spots are formed may be a slit-coating method, in which the liquid is applied on the substrate by flowing through slits, a spin-coating method, or the like. The atomizing method may be a spray method. The inactivating compound can be dissolved or dispersed in an appropriate solvent if required and then used in any of those methods.

Even if the inactivating compound is not dissolved in an appropriate solvent and applied as a liquid on the substrate, the inactivating substance may be sometimes appropriately applied on the substrate, on which spots are formed, by a vapor-contacting method such that the inactivating compound vaporizes when the inactivating compound is comparatively a low-boiling substance. The inactivating compound and the spot-formed substrate are hermetically placed in a chamber or a chamber with a heater and optionally heated to vaporize the inactivating compound, and then left standing for a certain period of time, thereby attaining the inactivation. Further, the atomizing treatment is particularly useful in the case of using the inactivating compound that does not vaporize at normal temperature (e.g., a polymeric compound or a polar compound having a strong intermolecular force). After dissolving the inactivating compound in an appropriate solvent, the inactivating compound can be directly sprayed on the spot-formed substrate using an atomizing device such as a spray.

In particular, the inactivation with the vaporized inactivating compound, any device capable of forming a vacuum space, such as a vacuum vapor deposition system or a simple vacuum desiccator made of polycarbonate, may be employed. In the case of using a liquid or solid inactivating compound having a high boiling point, the boiling point thereof can be lowered and the inactivating compound can vaporize even at relatively low temperature. Therefore, a method using such a device is effective in the case of a heat-labile inactivating compound

The atomizing treatment may be carried out such that a production line incorporated with a spray nozzle is constructed and the liquid containing the inactivating compound is then sprayed from the spray nozzle on the line, thereby coating the spot-formed substrate with the liquid containing the inactivating compound. At this time, the mist diameter of the liquid containing the inactivating compound ejected from the spray nozzle is about 10 μm to 20 μm, ideally 1 μm to 5 μm. This is because the trace of mist may remain on the substrate when the mist diameter is larger than the given range. In addition, the diameter of the liquid droplet containing the probe is about 50 μm to 500 μm. If the mist diameter is almost the same size as the diameter of the liquid droplet, the shape of the liquid droplet spotted on the substrate is physically changed, thereby leading to flow of the unreacted probe.

The inactivating compound should be selected as one having the capacity of inactivating the active group (X) of the unreacted probe. An inactivating compound suitably used in the present invention may be one that binds to and inactivates the active group (X) of the unreacted probe. The binding form of the inactivating compound to the probe may be a covalent bonding, an electrostatic bonding, a hydrophobic bonding, a van der Waals bonding, or the like.

Further, the inactivating compound may be any substance that satisfies the following three criteria.

(1) Any inactivated unreacted probe does not remain on the substrate or can be removed from the substrate.

(2) The functions of the probe immobilized on the spot area 501 cannot be damaged.

(3) The inactivating compound has a structure or characteristic features required for an inactivating treatment.

On the other hand, when the spot-formed substrate is subjected to an inactivating treatment but the inactivated unreacted probe is not removed from the substrate, further additional requirements for the inactivating compound are provided as following criteria in addition to the above criteria.

When the probe-immobilized carrier is produced without removing the inactivated probe, the inactivating compound requires the following criterion in addition to the above three criteria.

(4) The inactivating compound has a molecular structure that does not inhibit the reaction of a target substance in a specimen with the probe.

In this context, examples of the specific chemical structure other than the reactive group of the inactivating substance having inactivating actions, but not specifically limited to, include chemical structures containing an alkyl group, an alkoxyl group, a hydroxyl group, and a polyethylene glycol chain (PEG) and chemical structures having peptide chains (—NH—CO— bonds). The inactivating compounds include compounds as represented below, which may be used in combination of two or more if required.

(1) Examples of the inactivating compound when the active group X of the probe is an amino group and the reactive group Y of the inactivating compound is an N-hydroxy succinimidyl ester group are represented by the following chemical formulae:

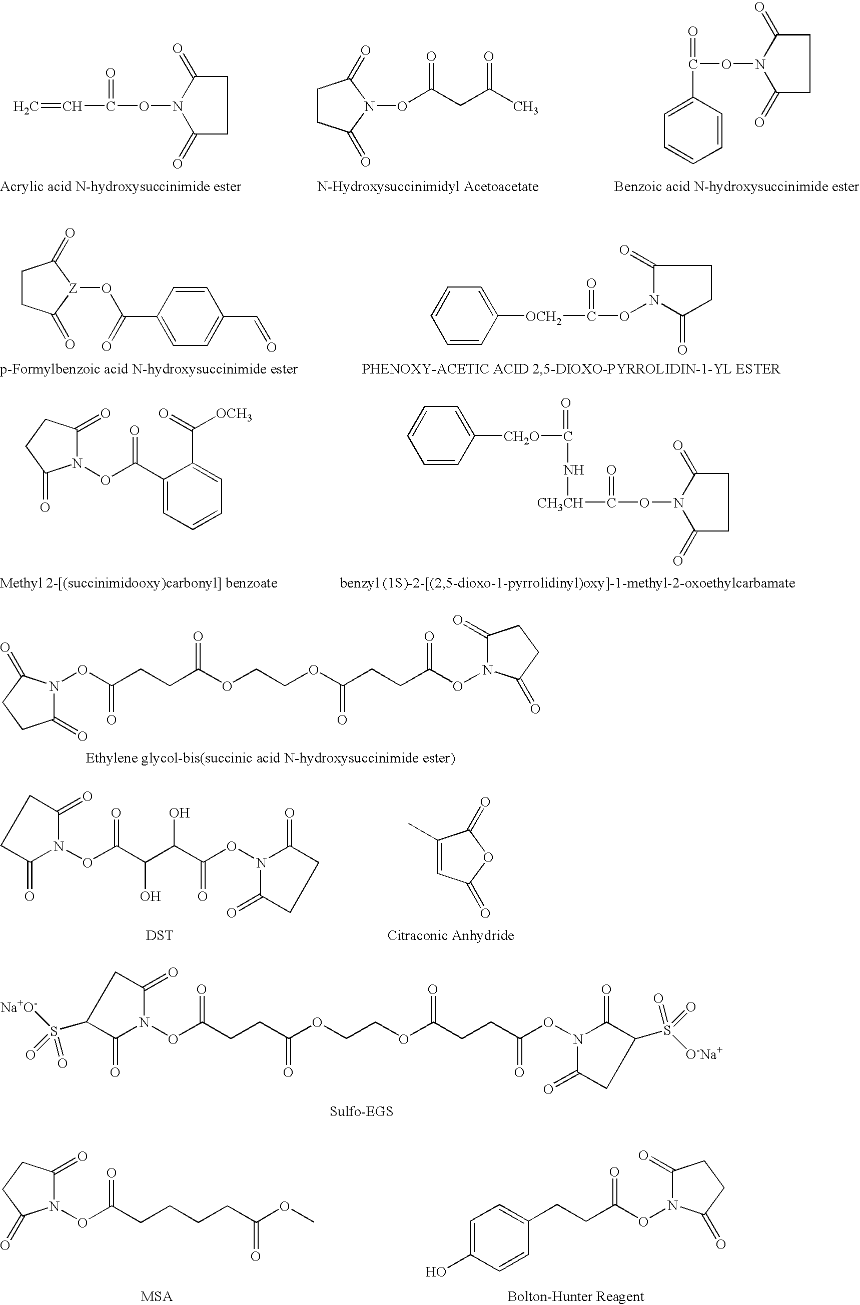

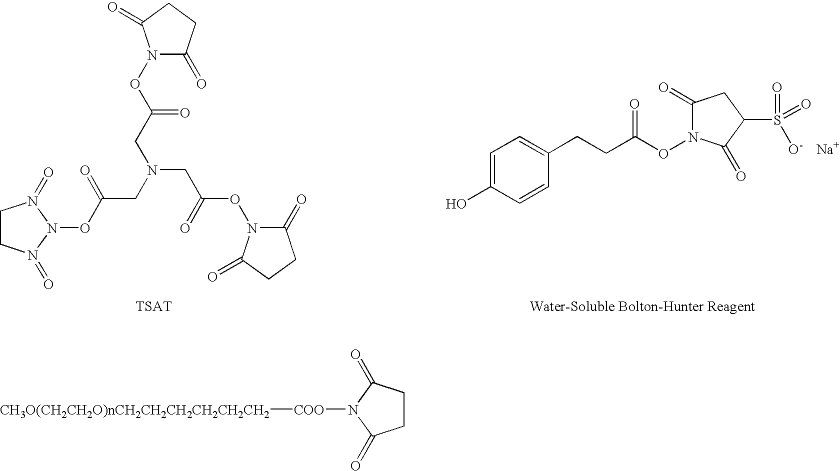

(2) Examples of the inactivating compound when the active group X of the probe is a thiol group and the reactive group Y of the inactivating compound is a maleimide group are represented by the following chemical formulae:

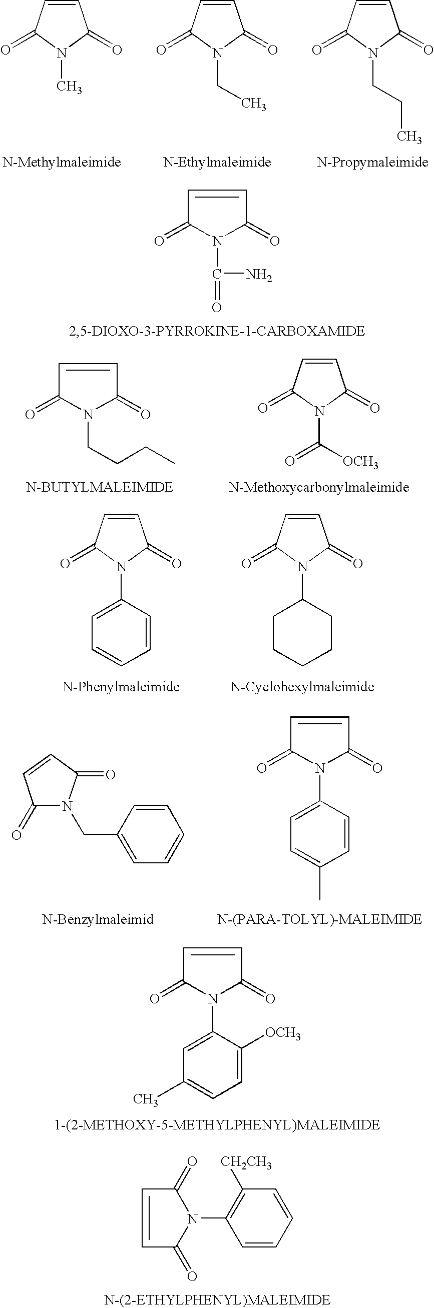

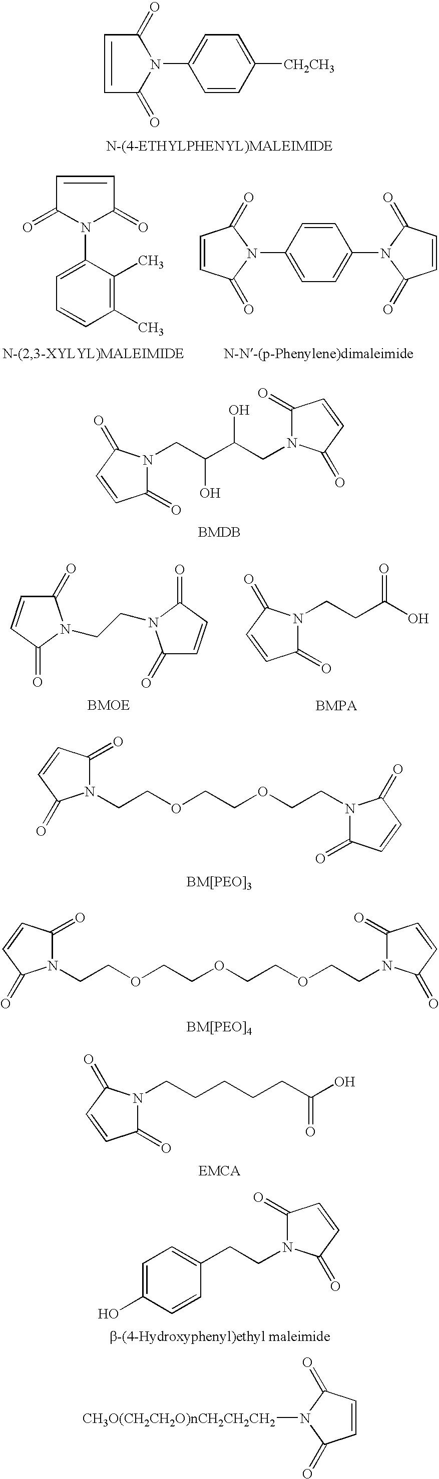

The substrate having spots formed thereon is used as a probe-immobilized carrier after the inactivating treatment is washed when needed. When the inactivated probe can be removed from the substrate, it is desirable to remove any inactivated unreacted probe as far as possible. Even the active group of the probe is inactivated, the probe itself (DNA portion in this example) may be sometimes adhered or stacked on the substrate through a relatively weak interaction, such as an electrostatic adsorption or van der Waals force. When the probe-immobilized carrier in which the unreacted probe is adhered or stacked on the substrate is used in a hybridization reaction with a specimen, the accuracy and sensitivity of the reaction can be decreased. This is because a hybridization reaction occurs competitively between the probe immobilized on the substrate and the unreacted probe as the probe itself keeps its activities to the target substrate even though it is inactive to a reactive substrate applied on the substrate. Further, the probe adhered or stacked on the substrate through the weak interaction may be sometimes peeled off by heat applied at the time of the hybridization reaction or by the action of a surfactant in the reaction solution. When the competitive hybridization reaction occurs, it becomes difficult to correctly estimate the amount of a target substance present in a specimen as a result of detecting the target substance. Therefore, it is desirable to remove the inactivated unreacted probe as far as possible. Here, a method of removing the inactivated unreacted probe may be carried out by washing with the solvent of a reaction solution employed in the hybridization reaction of the specimen with the probe-immobilizing carrier. Specifically, when the probe is a DNA probe, it is washed with a phosphate buffer, a tris-hydrochloric acid buffer, a tris-acetic acid buffer, a HEPES buffer, a MOPS buffer, a sodium acetate buffer, a sodium citrate buffer, or any of those optionally added with a salt such as sodium chloride, a chelating agent such as EDTA, an anionic surfactant such as SDS, or a non-ionic surfactant such as BriJ58, Nonident P-40, Triton X-100, Tween 20, Tween 80, etc.

Further, the probe-immobilized carrier after the inactivating treatment may be subjected to a blocking treatment for preventing a target substance from being adsorbed on the background, if required, before detecting the target substance or the like. The blocking treatment can be carried out, for example, by dipping the substrate in 1 to 2% by weight of bovine serum albumin in aqueous solution for 2 hours. The blocking treatment may be carried out, if required. For example, when a specimen is subjected to a hybridization reaction with the probe-immobilized carrier, the specimen may be spread over the respective spot areas 501 to a limited extent. Alternatively, it may not be carried out when the target substance in the specimen is not substantially adsorbed on the portion other than the spot.

### EXAMPLES

### Example 1

An inactivating treatment in the case of using thiol-labeled DNA probe will be described in detail in the order of the steps as follows.

(i) Synthesis of Probe and Binding of Target Substance to Fluorescent Label

A single-stranded DNA probe was used as a probe capable of specifically binding to a target substance. A DNA automatic synthesizer was used to synthesize a probe 1 having SEQ ID NO: 1 as described below. In addition, a mercapto (SH) group was introduced into the terminal of the single-stranded DNA of SEQ ID NO: 1 using Thiol-Modifier (manufactured by Glen Research Corp.) when synthesizing with the automated DNA synthesizer. Subsequently, the probe was collected after a normal deprotection and then purified by high-performance liquid chromatography.

Sequence of Probe 1

(SEQ ID NO: 1)

5′HS-(CH₂)₆-O-PO₂-O-ACTGGCCGTCGTTTTACA3′

Further, an unlabeled single-stranded DNA having a base sequence complementary to the sequence of the probe 1 as described above was synthesized by the automated DNA synthesizer and fluorescent substance Cy3 was then bound to the 5′ terminal, to thereby obtain a labeled single-stranded DNA probe.

(ii) Preparation of Probe-Immobilized Carrier

Washing of Substrate

The probe-immobilized carrier is prepared by immobilizing the probe on an appropriately chosen substrate. Here, as a substrate, a base plate made of synthetic quartz glass of a 2.54 cm (one-inch) by 7.62 cm (three-inch) square was used. The quartz glass base plate was washed as follows: brush-washing with purified water, rinsing with purified water, ultrasonic cleaning with alkaline detergent, rinsing with purified water, ultrasonic cleaning with purified water, rinsing with purified water, and drying with blowing nitrogen were carried out according to the conventional procedures, thereby preparing a quartz glass base plate having a cleaned surface.

Introduction of Reactive Substance into Substrate

Hereinafter, the step of introducing a reactive substance having a maleimide group capable of binding to a thiol group into quartz glass base plate.

An amino-silane coupling agent (trade name: KBM-603; manufactured by Shin-Etsu Chemical Co., Ltd.) was prepared and dissolved in water so as to be of 1 wt % and then stirred for 30 minutes to allow a methoxy group to be hydrolyzed. In the resulting aqueous solution, a quartz glass base plate which had been washed was dipped for 30 minutes (heated at 80° C. in hot bath) and then pulled out and washed with purified water, followed by subjecting to baking treatment at 120° C. for 1 hour in an oven. Subsequently, 2.7 mg of N-maleimido-caproyloxysuccinimide (manufactured by DOJINDO LABORATORIES, hereinafter abbreviated as EMCS) was weighed and dissolved in a dimethylsulfoxide (DMSO)/ethanol (1:1) solution so as to be of a final concentration of 0.3 mg/ml, thereby preparing an EMCS solution. The quartz glass base plate which had been subjected to the baking treatment, was dipped in the EMCS solution for 2 hours at room temperature to introduce a maleimide group on the surface of the quartz glass base plate. After the treatment with the EMCS solution, the base plate was sequentially washed with a DMSO/ethanol mixture solution and ethanol and then dried under nitrogen atmosphere.

Immobilization of Probe

A probe solution for inkjet including a single-stranded DNA probe fragment with the sequence of probe 1 (SEQ ID NO: 1) that is synthesized in the above-mentioned step (i) was prepared. An aqueous solution containing 7.5 wt % of glycerin, 7.5 wt % of urea, 7.5 wt % of thiodiglycol, and 1.0 wt % of acetylene alcohol (trade name: Acetylenol E100, manufactured by Kawaken Fine Chemicals Co., Ltd.) was used as a solvent. Five types of the aqueous solution were prepared so that the probe concentrations thereof were 8.75, 26.25, 43.75, 61.25, and 87.5 μM, respectively. It has been known that the saturated concentration of the thiol-labeled probe in reaction is approximately 50 μM with respect to the amount of a maleimide group on the quartz glass base plate. Aqueous solutions containing probes at different concentrations were spotted on the substrate treated with an EMCS solution by the inkjet method. After spotting on the substrate treated with the EMCS solution, the spot-formed quartz glass base plate was placed in a chamber with constant temperature and humidity for 30 minutes to immobilize probes in the respective probes on the substrate, thereby resulting in a probe-immobilized carrier.

(iii) Inactivation of Unreacted DNA Probe Labeled with Thiol

The inactivating treatment, which is a characteristic feature of the present invention, will be described in detail with reference to one example.

Maleimido-PEGs (trade name: SUNBRIGHT MEMAL-50, manufactured by NOF) was used as an inactivating compound to carry out an inactivating treatment on an unreacted probe. The compound has a molecular weight of 5,000 and includes a maleimide group in the molecule thereof, which can be covalently bound to a thiol group, the active group of the DNA probe. The structural formula of the compound is represented below.

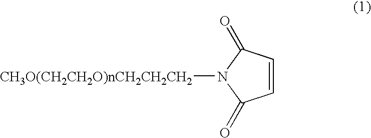

A solution containing Maleimido-PEGs and capable of being ejected by the inkjet method was prepared. An aqueous solution containing 7.5% by weight of glycerin, 7.5% by weight of urea, 7.5% by weight of thiodiglycol, and 1.0% by weight of acetylene alcohol (trade name: Acetylenol E100, manufactured by Kawaken Fine Chemicals) was used as a solvent. Further, the concentration of Maleimido-PEGs in the aqueous solution was set to 100 μM.

Subsequently, the solution containing the inactivating compound thus prepared was spotted on the probe-immobilizing carrier prepared in the above step (ii). After that, the substrate was placed in a chamber with constant temperature and humidity for 30 minutes to inactivate the thiol group of the unreacted probe.

Further, regarding an inkjet head used in the inkjet method, one used for applying the probe solution to the substrate is of the same specification as that of one used for applying the inactivating compound as well as equal to be amount of the liquid droplet ejected (about 8 pl).

(iv) Removal of Unreacted Probe

Subsequently, the substrate was washed with a NaCl/50 mM phosphate buffer (pH 7.0) and then lightly washed with pure water to remove the unreacted probe. The substrate was dried by drying with nitrogen blowing, thereby obtaining a probe-immobilized carrier. In this case, for comparison, an unreacted probe was removed from the probe-immobilized carrier prepared in the above step (ii) without carrying out the inactivating treatment of the above step (iii), thereby preparing a probe-immobilized carrier.

(v) Hybridization Reaction and Fluorescent Evaluation

A target substance prepared in the above step (i) and fluorescently labeled was dissolved in a NaCl/50 mM phosphate buffer (pH 7.0) so as to be a final concentration of 5 nM. A probe-immobilized carrier prepared in the presence of the inactivating treatment as described above and a probe-immobilization carrier prepared in the absence of the inactivating treatment were respectively dipped in the solution to carry out a hybridization reaction for 2 hours at 45° C. Subsequently, the probe-immobilized carrier was washed with a NaCl/50 mM phosphate buffer (pH 7.0) and then washed lightly with pure water to remove a salt content, followed by drying the probe-immobilized carrier with nitrogen blowing. The fluorescence intensity of the background area 502 of the dried probe-immobilizing carrier was measured using a fluorescence scanner (trade name: GenePix 4000B, manufactured by Axon Instruments, Inc.). The same measurement conditions were employed in both the examples and the comparative examples (wavelength used in the measurement of fluorescence strength is 532 nm).

(vi) Results

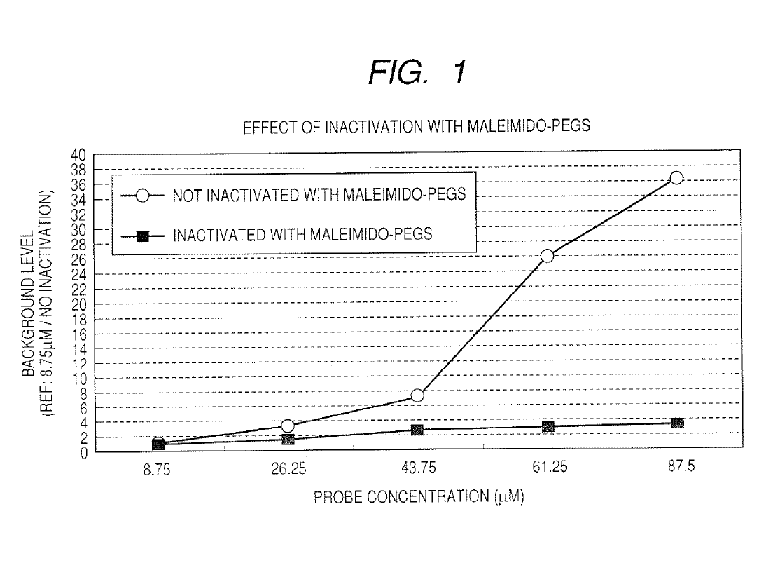

The average fluorescence intensity on the background area 502 in the presence or absence of the inactivating treatment with Maleimido-PEGs was obtained by plotting with respect to the concentration of the probe in the probe solution. The results were shown in FIG. 1. Further, the standard for the intensity of fluorescence generated from the background area 502 was a probe-immobilized carrier prepared under the conditions with a lowest probe concentration of 8.75 μM in the absence of the inactivating treatment. Regarding FIG. 1, when the probe-immobilized carrier prepared in the absence of the inactivating treatment was used, a sudden increase in background was observed at about 61.25 μM, which exceeded 50 μM of the saturated concentration of the probe (drastic flow of spots in a fluorescent image was confirmed in a fluorescent image not shown in the figure). On the other hand, when the probe-immobilized carrier prepared in the presence of the inactivating treatment was used, it was found that no increase in background was confirmed even using a probe solution of 61.25 μM or more. As a result, the inactivating treatment inactivated the unreacted probe and prevented the probe from being immobilized on the background area.

### Example 2

In this example 2, an inactivating treatment using an amino-labeled DNA probes will be described in detail in the order of steps as follows:

(i) Preparation of Probe and Binding of Target Substance to Fluorescent Label

A single-stranded DNA was used as a probe capable of specifically binding to a target substance. A DNA automatic synthesizer was used to synthesize a probe 2 having SEQ ID NO: 2 as described below. An amino (NH₂) group was introduced into the end of the terminal of the single-stranded DNA probe using Amino-Modifier (manufactured by Glen Research Corp.) when synthesized with the DNA automatic synthesizer. Subsequently, the probe was collected after normal deprotection and then purified by high-speed liquid chromatography.

Sequence of probe 2:

(SEQ ID NO: 2)

5′NH₂-(CH₂)₆-PO₂-O-ACTGGCCGTCGTTTTACA3′

In addition, the single-stranded DNA probe having the sequence of the probe as described above was synthesized on a DNA automatic synthesizer. A fluorescent substance Cy3 was then bound to the 5′ terminal of the synthesized single-stranded DNA, to thereby obtain a labeled single-stranded DNA probe.

(ii) Preparation of Probe-Immobilized Carrier

Washing of Base Plate

The probe-immobilized carrier is prepared in the same manner as Example 1, by immobilizing the probe on an appropriately chosen substrate. Here, as a substrate, a quartz glass base plate of a 2.54 cm (one-inch) by 7.62 cm (three-inch) square was used. The quartz glass base plate was washed in the same manner as in Example 1 by the following methods. That is, brush-washing with purified water, rinsing with purified water, ultrasonic cleaning with alkaline detergent, rinsing with purified water, ultrasonic cleaning with purified water, rinsing with purified water, and drying with nitrogen blowing were carried out, to thereby prepare a quartz glass base plate having a cleaned surface.

Introduction of Reactive Substance into a Substrate

The process of introducing a reactive substance made from an epoxy group capable of being bound to amino group into a quartz glass base plate will be described in detail below. A 50 wt % methanol aqueous solution containing 1 wt % of a silane-coupling agent (trade name: KBM403, manufactured by Shin-Etsu Chemical Co., Ltd.) including a silane compound (Y-glycidoxypropyl-trimethoxysilane) having an epoxy group was stirred for 3 hours at room temperature. With this stirring, a methoxy group in the silane compound was hydrolyzed and the solution was applied on the surface of the base plate using a spin coater, heated at 100° C. for 5 minutes, and dried to introduce an epoxy group into the surface of the base plate.

Immobilization of Probe

An amino-labeled DNA probe previously prepared was dissolved in a 50 mM NaCl buffer solution (pH 8) so as to be 200 μM in final concentration, thereby obtaining a probe solution containing the amino-labeled DNA probe. Separately, a single-stranded DNA probe made of a complementary sequence of SEQ ID NO: 2 was dissolved in a 50 mM NaCl buffer solution (pH 8) so as to be 200 μM in final concentration, thereby obtaining a probe solution containing the single-stranded DNA probe which was unlabeled with an amino group. Then, 100 μM of the probe solution containing the unlabeled DNA probe was added to 100 μM of the probe solution containing the amino-labeled DNA probe. Further, the mixture was cooled down from 90° C. to 25° C. for 2 hours to form a hybrid of each DNA probe and each single-stranded nucleic acid. Sequentially, the solution containing the hybrid was added to an aqueous solution containing 7.5% by weight of glycerin, 7.5% by weight of urea, 7.5% by weight of thiodiglycol, and 1.0% by weight of acetylene alcohol (trade name: Acetylenol E100, manufactured by Kawaken Fine Chemicals). Seven different aqueous solutions were prepared so that final concentrations of the hybrid products in the respective aqueous solutions reached to 8, 24, 40, 56, 72, 88, and 104 μM. Here, it was found that the reaction-saturated concentration of the amino-labeled probe was about 65 μM with respect to the substance quantity of epoxy group on the substrate.

These seven different probe solutions were spotted on the surface of a substrate, in which an epoxy group was introduced, and then placed in a chamber with constant temperature and humidity for 12 hours, thereby obtaining a probe-immobilized carrier. By the way, the amino group in the probe itself forms a hybrid with a complete complementary single-stranded DNA probe, so it cannot react with the epoxy group on the surface of the substrate.

(iii) Inactivation of Unreacted Amino-Labeled DNA Probe

A compound of the formula (2), Carboxyl-PEGs (NHS active esters) (trade name: SUNBRIGHT ME-020AS, manufactured by NOF) was used as an inactivating compound to inactivate an unreacted DNA in a manner similar to Example 1. The compound has a molecular weight of about 5,000 and an NHS active ester group in molecule, capable of covalently binding to the amino group, which is the active group of the DNA probe. The structure of the compound is as follows:

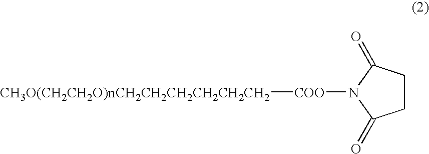

A solution containing Carboxyl-PEGs (NHS active esters) and capable of being ejected by the inkjet method was prepared. The solvent used was an aqueous solution containing 7.5% by weight of glycerin, 7.5% by weight of urea, 7.5% by weight of thiodiglycol, and 1.0% by weight of acetylene alcohol (trade name: Acetylenol E100, manufactured by Kawaken Fine Chemicals). Further, the concentration of Carboxyl-PEGs in the aqueous solution was set to 100 μM.

Subsequently, the solution containing the inactivating compound thus prepared was spotted on the probe-immobilizing carrier prepared in the above step (ii). After that, the substrate was placed in a chamber with constant temperature and humidity for 30 minutes to inactivate the amino group of the unreacted probe.

Further, regarding an inkjet head used in the inkjet method, one used for applying the probe solution to the substrate is of the same specification as that of one used for applying the inactivating compound as well as equal to the amount of the liquid droplet ejected (about 8 pl)

(iv) Removal of Unreacted Probe

Subsequently, the substrate was washed with pure water at 80° C. for 10 minutes. The complementary chain forming the hybrid product with the probe was dissociated from the probe and then washed out, while the unreacted probe subjected to the inactivated treatment was simultaneously washed out. After that, the substrate was dried by nitrogen blowing, thereby obtaining a probe-immobilized carrier for hybridization.

In this case, for comparison, an unreacted probe was removed from the probe-immobilized carrier prepared in the above step (ii) without carrying out the inactivating treatment of the above step (iii), thereby preparing a probe-immobilized carrier.

(v) Hybridization Reaction and Fluorescent Evaluation

A target substance prepared in the above step (i) and fluorescently labeled was dissolved in a NaCl/50 mM phosphate buffer (pH 7.0) so as to be a final concentration of 5 nM. A probe-immobilized carrier prepared in the presence of the inactivating treatment as described above and a probe-immobilization carrier prepared in the absence of the inactivating treatment were respectively dipped in the solution to carry out a hybridization reaction for 2 hours at 45° C. Subsequently, the probe-immobilized carrier was washed with a NaCl/50 mM phosphate buffer (pH 7.0) and then washed lightly with pure water to remove a salt content, followed by drying the probe-immobilized carrier with nitrogen blowing. The fluorescence intensity of the background area 502 of the dried probe-immobilizing carrier was measured using a fluorescence scanner (trade name: GenePix 4000B, manufactured by Axon Instruments, Inc.). The same measurement conditions were employed in both the examples and the comparative examples (wavelength used in the measurement of fluorescence strength is 532 nm).

(6) Results

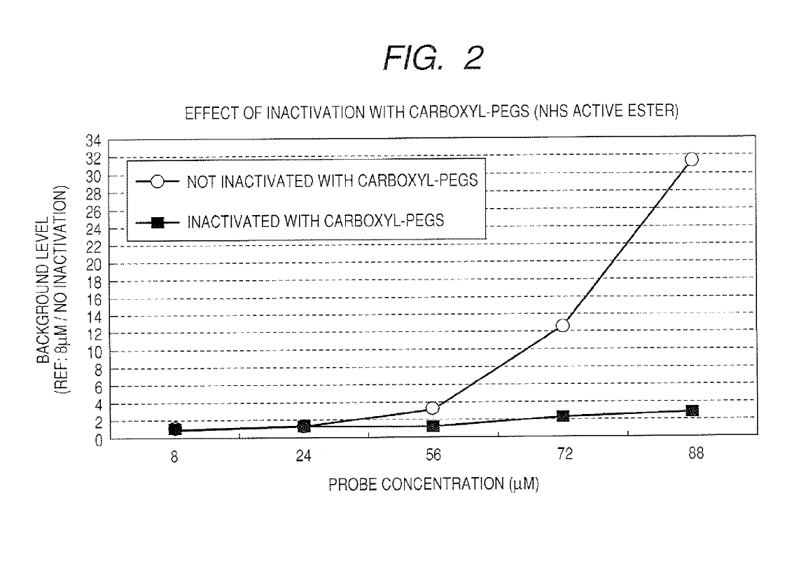

The average fluorescence intensity on the background area 502 in the presence or absence of the inactivating treatment with Carboxyl-PEGs (NHS active ester) was obtained by plotting with respect to the concentration of the probe in the probe solution. The results were shown in FIG. 2. Further, the standard for the intensity of fluorescence generated from the background area 502 was a probe-immobilized carrier prepared under the conditions with a lowest probe concentration of 8 μM in the absence of the inactivating treatment. Regarding FIG. 2, when the probe-immobilized carrier prepared in the absence of the inactivating treatment was used, a sudden increase in background was observed at about 72 μM, which exceeded 65 μM of the saturated concentration of the probe (drastic flow of spots in a fluorescent image was confirmed in a fluorescent image not shown). On the other hand, when the probe-immobilized carrier prepared in the presence of the inactivating treatment was used, it was found that no increase in background was confirmed even using a probe solution of saturated concentration or more. Therefore, it was confirmed that the inactivating treatment inactivated the unreacted probe and prevented the probe from being immobilized on the background area.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2006-135029, filed May 15, 2006, which is incorporated herein by reference in its entirety.

What is claimed is:

1. A method of manufacturing a probe-immobilized carrier in which a probe capable of specifically binding to a target is immobilized on a substrate, comprising the steps of:
(1) coating the substrate with a reactive substance having a reactive group for immobilizing the probe on the substrate;
(2) applying the probe to a surface of the substrate coated with the reactive substance;
(3) immobilizing the applied probe on the substrate; and
(4) applying an inactivating compound capable of inactivating the probe to a probe-immobilized area on the substrate to inactivate an unreacted probe remaining on the substrate in the probe-immobilized area.

2. A method of manufacturing a probe-immobilized carrier according to claim 1, further comprising the step of removing the inactivated probe from the substrate.

3. A method of manufacturing a probe-immobilized carrier according to claim 1, further comprising spotting a liquid containing the inactivating compound on the substrate in the probe-immobilized area in which the unreacted probe remains.

4. A method of manufacturing a probe-immobilized carrier according to claim 3, wherein the spotting is carried out by an inkjet method.

5. A method of manufacturing a probe-immobilized carrier according to claim 1, further comprising spraying a liquid containing the inactivating compound on the substrate in the probe-immobilized area in which the unreacted probe remains.

6. A method of manufacturing a probe-immobilized carrier according to claim 1, further comprising applying a liquid containing the inactivated compound on the substrate by flowing the liquid through a slit.

7. A method of manufacturing a probe-immobilized carrier according to claim 1, further comprising bringing a gas containing the inactivating compound into contact with the substrate.


SEQUENCE LISTING (2 sequences)

SEQ ID NO: 1 - DNA, 18 bp, Artificial
  Thiol-labeled DNA probe
actggccgtcgttttaca

SEQ ID NO: 2 - DNA, 18 bp, Artificial
  Amino-labeled DNA probe
actggccgtcgttttaca